(19) United States
(12) Patent Application Publication
(10) Pub. No.: US 2025/0288502 A1
(43) Pub. Date: Sep. 18, 2025

(54) UV FILTERS AND COSMETIC COMPOSITIONS RELATED THERETO

(71) Applicant: ADVANCED DISPERSED PARTICLES, S.L., Madrid (ES)

(72) Inventors: Blanca MOTOS PÉREZ, Madrid (ES); Antonio Ismael SANTAMARÍA JIMÉNEZ, Madrid (ES); Irene ZALDÍVAR NOTARIO, Madrid (ES)

(21) Appl. No.: 18/945,676

(22) Filed: Nov. 13, 2024

(30) Foreign Application Priority Data
Mar. 12, 2024 (EP) 24382265.7

(51) Int. Cl. A61K 8/02; A61K 8/19; A61K 8/27; A61K 31/455; A61K 36/185; A61K 36/23; A61P 29/00; A61Q 17/04

(52) U.S. Cl. CPC A61K 8/0275; A61K 8/19; A61K 8/27; A61K 31/455; A61K 36/185; A61K 36/23; A61P 29/00; A61Q 17/04; A61K 2800/61; A61K 2800/652

(57) ABSTRACT

The present invention refers to a novel hierarchical composite material comprising (a) ZnO nanoparticles having a mean particle of less than 100 nm, and (b) ZnO support particles having a mean particle size of more than 100 nm; wherein said nanoparticles are dispersed onto the support particles. The invention further refers to cosmetic compositions comprising such material, methods of preparation and uses thereof.

### CROSS REFERENCE TO RELATED APPLICATIONS

This application is filed under 35 U.S.C. § 119(a) and claims priority to European Patent Application No. EP24382265.7, filed 12 Mar. 2024, which is incorporated herein by reference in its entirety.

### FIELD OF THE INVENTION

The present invention relates generally to UV filter composite materials based on zinc oxide for sunscreen cosmetic preparations.

### BACKGROUND

The solar radiation reaching our skin involves three components: UV, Visible (Vis) and IR light. Their depth of penetration and damage depends on their wavelength and energy.

The UVB radiation (290-320 nm) acts mainly on the epidermis, the most superficial layer. It causes erythema (sunburn) and partially penetrates the dermis. The UVA radiation (320-400 nm) passes through the dermis and is responsible for photo-ageing and pigment darkening. Both UVB and UVA radiations induce delayed tanning and are associated with cellular damage, through direct or indirect DNA damage, and with potential risk of skin cancer.

The effects of visible light (380 to 700 nm) and IR radiation (700 nm to 1 mm) are less known. However, it has been reported that Vis light can induce pigmentation, especially in skin types IV-VI, and that this is darker and more sustained than pigmentation induced by UVA light. The severity of photo-induced pigmentary disorders, such as melasma or post inflammatory hyperpigmentation may be related to the exposure to Vis, which can also induce the generation of reactive species oxygen (ROS).

Therefore, there has been an increasing interest in recent years in cosmetics that protect against the different components of the solar spectrum and not only UV radiation (SPF30-50).

Titanium dioxide and zinc oxide are Generally Recognized As Safe and Effective (GRASE) for use in OTC sunscreens to protect from UV radiation. As TiO₂ is more effective in UVB and ZnO in the UVA range, both are usually combined to ensure a broad-band UV protection. These mineral filters are commonly marketed as nanoparticles to minimize whiteness on the skin. However, the more transparent (less visible) the filter is on the skin, the less effective the protection against UVA, Vis and IR radiation. Besides, increasing the number of ingredients in the formula can hinder and lengthen the development time of a cosmetic product.

Thus, there remains a need in the art to develop cosmetic products that are simpler and stable, and to achieve protection in different areas of the solar spectrum.

### BRIEF DESCRIPTION OF THE INVENTION

The authors of the present invention have developed a novel material with unexpectedly high sun protection factor (SPF) values for a UV-filter composed of zinc oxide (ZnO), which provides excellent UVA and UVB balance protection with a single filter in the formula.

Advantageously, this material shows excellent stability and versatility upon incorporation to a sunscreen emulsion formulation. More particularly, the UV-filter can be incorporated to either the oil or the aqueous phase and maintain the initial effectiveness without showing phase migration of the zinc oxide or pH variations.

Therefore, a first aspect of the invention is directed to a hierarchical composite material comprising:
a) zinc oxide nanoparticles having a mean particle size of less than 100 nm, and
b) zinc oxide support particles having a mean particle size between 120 and 800 nm;
wherein said nanoparticles are dispersed onto the support particles.

In a second aspect, the present invention is directed to a cosmetic composition comprising the hierarchical composite material of the first aspect.

In a third aspect, the present invention is directed to a method for the preparation of the hierarchical composite material of the first aspect of the invention, said method comprising the steps of:
i) providing ZnO support particles with a mean size between 120 and 800 nm,
ii) adding ZnO nanoparticles with a mean size of less than 100 nm to said support particles, and
iii) stirring in a dry medium to disperse the nanoparticles onto the support particles at a speed from 5 to 100 rpm.

In a fourth aspect, the present invention is directed to a hierarchical composite material obtainable by the method of the third aspect.

A further aspect relates to a method of filtering UVA and UVB radiation, said method comprising the topical application of the composite material as defined in the first or third aspect, or the cosmetic formulation as defined in the second aspect to a subject in need thereof.

In a sixth aspect, the invention relates to a method of treatment or prevention of skin inflammation, said method comprising administering an effective amount of the hierarchical composite material of the invention to a subject in need thereof.

### BRIEF DESCRIPTION OF THE DRAWINGS

FIG. 1: UV-Vis-IR absorption spectra of (a) ZnZn-17% NP, (b) ZnZn-20% NP, (c) ZnZn-22.5% NP, (d) ZnZn-25% NP and (e) ZnZn-30% NP materials.

FIG. 2. UV-Vis-IR absorption spectra of (a) ZnZn-light, (b) ZnZn-medium and (c) ZnZn-warm materials vs (d) ZnZn-25% NP material as reference.

FIG. 3. UV-Vis-IR absorption spectra of (a) ZnZn-niacinamide, vs (b) ZnZn-25% NP material as reference.

FIG. 4. UV-Vis-IR absorption spectra of (a) ZnZn-argania, vs (b) ZnZn-25% NP material as reference.

FIG. 5. UV-Vis-IR absorption spectra of (a) ZnZn-kaolin, vs (b) ZnZn-25 material as reference.

FIG. 6. SEM powder image of ZnZn-17% NP material, 300 nm scale.

FIG. 7. SEM powder image of ZnZn-20% NP material, 300 nm scale.

FIG. 8. SEM powder image of ZnZn-22.5% NP material, 300 nm scale.

FIG. 9. SEM powder image of ZnZn-22.5% NP material, 3 μm scale.

FIG. 10. SEM powder image of ZnZn-25% NP material, 300 nm scale.

FIG. 11. SEM powder image of ZnZn-30% NP material, 300 nm scale.

FIG. 12. SEM powder-in-ethanol image of ZnZn-25% NP material, 200 nm scale.

FIG. 13. SEM powder image of ZnZn-light material, 300 nm scale.

FIG. 14. SEM powder image of ZnZn-medium material, 300 nm scale.

FIG. 15. SEM powder image of ZnZn-warm material, 300 nm scale.

FIG. 16. SEM powder-in-ethanol image of ZnZn-warm material, 100 nm scale.

FIG. 17. SEM powder image of ZnZn-niacinamide material, 300 nm scale.

FIG. 18. SEM powder image of ZnZn-niacinamide material, 100 μm scale.

FIG. 19. SEM powder image of ZnZn-carrot material, 300 nm scale.

FIG. 20. SEM powder image of ZnZn-argania material, 300 nm scale.

FIG. 21. SEM powder image of ZnZn-argania material, 3 μm scale.

FIG. 22. SEM powder-in-ethanol image of ZnZn-argania material, 100 nm scale FIG. 23. SEM powder image of ZnZn-caolin material, 300 nm scale.

FIG. 24. Cryo-TEM image of ZnZn-25% NP material.

FIG. 25. IL-6 secretion of healthy skin, photo-aged skin or irradiated and treated with ZnZn-25, ZnZn-niacinamide or ZnZn-argania.

FIG. 26. IL-8 secretion of healthy skin, photo-aged skin or irradiated and treated with ZnZn-25, ZnZn-niacinamide

### DETAILED DESCRIPTION OF THE INVENTION

The present invention provides for novel hierarchical composite materials which exhibit outstanding properties as sunscreen agents. In addition, the ZnO composite material of the invention provide for sunscreen formulations with excellent stability. The present invention also provides methods for obtaining the composite material of the invention and describes uses thereof.

In one aspect, the present invention relates to a hierarchical composite material comprising:
(a) ZnO nanoparticles having a mean particle size of less than 100 nm, and
(b) ZnO support particles having a mean particle size between 120 and 800 nm;
   wherein said nanoparticles are dispersed onto the support particles.

As used herein, the term “hierarchical” in relation to the composite material of the present invention refers to a material having structures spanning different sizes, i.e., particles having a largest dimension of a size of 100 nm or less and particles having a largest dimension of a size of more than 100 nm and up to 3 microns.

For the purposes of the present invention, the term “nanoparticle” generally refers to a particle having a largest dimension of a size of 100 nm or less, such as of between 1 and 100 nm.

In a particular embodiment, the ZnO nanoparticles are present in a percentage of 15 to 37% by weight with respect to the total weight of the composite material. Preferably, the ZnO nanoparticles are present in a percentage of 17% to 35% by weight with respect to the total weight of the composite material. More preferably, the ZnO nanoparticles are present in a 19% to 30% by weight, more preferably 20% to 28% with respect to the total weight of the composite material.

The ZnO nanoparticles may be uncoated or coated with triethoxycaprylylsilane, polyhydroxystearic acid, stearic acid or stearate, stearoyl glutamic acid, dimethicone, hydrogen dimethicone or other dimethicone derivates, silica, hydrated silica, alumina, aluminum hydroxide or a combination thereof. Preferably, the ZnO nanoparticles are uncoated.

The ZnO nanoparticles of the material according to the present invention have a mean particle size of less than 100 nm. In a particular embodiment, the ZnO nanoparticles have a mean particle of less than 90 nm, less than 80 nm, less than 70 nm, less than 60 nm. In a particular embodiment, the ZnO nanoparticles have a mean particle size of more than 1 nm, more than 5 nm, more than 10 nm, more than 20 nm. In a further particular embodiment, the ZnO nanoparticles have a mean particle size between 20 and 99 nm, preferably between 30 and 80, more preferably between 40 and 70.

The term “particle size” as used herein means the diameter of the particle if the particle is spherical or, if the particle is non-spherical, the volume-based particle size. The volume-based particle size is the diameter of the sphere that has the same volume as the non-spherical particle in question. Particle size as described herein can be determined by various conventional methods of analysis, such as SEM, TEM, Laser light scattering or laser diffraction, dynamic light scattering, centrifugal particle size. The size of all particles analyzed is typically expressed as a mean value. For example, nanoparticles, e.g. ZnO nanoparticles, mean particle size may be determined centrifugal particle size technique and support particles, e.g. ZnO support particles, mean particle size may be determined by Scanning Electron Microscopy.

The ZnO nanoparticles of the material of the present invention may have any morphology, such as spherical, polyhedral, laminar, fibrillar or irregular. Preferably, the ZnO nanoparticles are spherical.

The ZnO support particles of the material of the present invention act as a support for the ZnO nanoparticles. Without wishing to be bound by any theory, the authors of the present invention believe that the ZnO nanoparticles are dispersed on the surface of the ZnO support particles and anchored thereto by short-range forces such as van der Waals forces, hydrogen bonds. The ZnO support particles of the present invention may also act as a support for further components of the composite material, when present.

The ZnO support particles may be uncoated or coated with triethoxycaprylylsilane, polyhydroxystearic acid, stearic acid or stearate, stearoyl glutamic acid, dimethicone, hydrogen dimethicone or other dimethicone derivates, silica, hydrated silica, alumina, aluminum hydroxide or a combination thereof. Preferably, the ZnO support particles are uncoated.

The ZnO support particles of the material of the present invention have a mean size of more than 100 nm. In a particular embodiment, the ZnO support particles have a mean size of more than 150 nm, more than 200 nm, more than 300 nm, more than 400 nm, more than 500 nm. In a particular embodiment, the ZnO support particles have a mean size of less than 1000 nm, less than 900 nm, less than 800 nm, less than 700 nm, less than 600 nm. In a further particular embodiment, the ZnO support particles have a mean particle size between 120 and 800 nm, preferably between 135 and 700 nm, more preferably between 150 and 600 nm, even more preferably between 200 and 400 nm.

The ZnO support particles of the material of the present invention may have any morphology, such as spherical, polyhedral, laminar, fibrillar or irregular. Preferably, the ZnO support particles are mixtures of polyhedral and laminar morphologies

In a particular embodiment, the weight ratio of ZnO nanoparticles:ZnO support particles in the material is from about 0.18 to about 0.60, preferably from about 0.2 to about 0.6, more preferably from about 0.25 to about 0.45, even more preferably from about 0.29 to about 0.38.

The composite material of the present invention may further comprise one or more other cosmetic ingredients suitable for use in contact with human skin without undue toxicity, incompatibility, instability, allergic response, and the like within the scope of sound medical judgment and in accordance with Regulation (EC) No 1223/2009 of the European Parliament and of the Council of 30 Nov. 2009 on cosmetic products. The CTFA Cosmetic Ingredient Handbook, Second Edition (1992) describes a wide variety of non-limiting cosmetic ingredients commonly used in the skin care industry, which are suitable for use in the compositions of the present invention. Examples of these ingredients include, without limitation, abrasives, absorbents, additives, anticorrosives, antidandruff and antifoaming agents, antimicrobials, antioxidants, antiperspirant and antistatic agents, binders, bleaching agents, botanicals, binders, biological additives, bleaching agents, botanicals, buffering agents, chelating agents, cosmetic colorants (including pigments), denaturants, deodorant agents, depilatory agents, emollients, emulsifying agents, emulsion stabilizers, film formers, hair dyes, humectants, opacifiers, oral care agents, oxidizing agents, preservatives, propellants, reducing agents, solvents, surfactants, UV absorbers, viscosity controlling agents.

In any embodiment of the present invention, however, the cosmetic ingredients useful herein can be categorized by their chemical nature, i.e., organic or inorganic ingredients, or by the benefit they provide. However, it is to be understood that the cosmetic ingredients useful herein can in some instances provide more than one benefit. Therefore, classifications herein are made for the sake of convenience and are not intended to limit the active to that particular application or applications listed.

In a particular embodiment, the composite material further comprises a pigment. Any suitable pigment known in the art may be used. Non-limiting examples of cosmetic pigments include inorganic pigments such as metal oxides, for example, black, red and yellow iron oxides, titanium dioxide and zinc oxide, cobalt aluminum oxide, chromium oxide or hydroxide such as salts, for example silicates, phosphates, aluminium, zinc, magnesium and calcium stearates, aluminium hydroxide, calcium and barium sulfate, ultramarine or manganese violet; and organic pigments such as true pigments, toners, lakes or carbon black or other of natural origin as beta-caroten and derivates or carotenoids. Detailed information of cosmetic pigments can be found in Regulation (EC) No 1223/2009 of the European Parliament and of the Council of 30 Nov. 2009 on cosmetic products.

In preferred embodiments, the pigment is iron oxide, such as iron oxide particles. Preferably, the iron oxide is selected from the group consisting of iron(III) oxide-hydroxide (FeOOH), iron(III) oxide or red iron oxide (Fe₂O₃), iron(II,III) oxide or black iron oxide (Fe₃O₄) and combinations thereof. The iron oxide particles may have any suitable morphology known in the art, such as spherical, polyhedral, rod-like, irregular or mixtures thereof.

In a particular embodiment, the iron oxide particles have a mean size between 10 nm and 2000 nm, preferably a mean size between 30 nm and 1000 nm, more preferably between 50 nm and 500 nm. The particle size may be determined by high-resolution electron microscopy.

The iron oxide particles may be uncoated or coated with organic compounds as silicone or silane or inorganic compounds as stearate or a combination thereof. Preferably, the iron oxide particles are uncoated.

In a particular embodiment, the iron oxide particles are comprised in an amount from about 0.1 to about 15%, preferably from about 1% to about 10%, more preferably from about 2% to 8% by weight with respect of the total weight of the composite material.

In a more particular embodiment, the iron oxide is FeOOH, preferably in an amount from about 2% to 8% by weight with respect of the total weight of the composite material. In another more particular embodiment, the iron oxide is Fe₂O₃, preferably in an amount from about 0.2% to about 2% by weight with respect of the total weight of the composite material. In another more particular embodiment, the iron oxide is Fe₃O₄, preferably in an amount from about 0.1% to about 1% by weight with respect of the total weight of the composite material.

In another particular embodiment, the iron oxide is a combination of FeOOH, preferably in an amount from about 2% to 8% by weight with respect of the total weight of the composite material, Fe₂O₃, preferably in an amount from about 0.2% to about 2% by weight with respect of the total weight of the composite material, and/or Fe₃O₄, preferably in an amount from about 0.1% to about 1% by weight with respect of the total weight of the composite material.

In another particular embodiment, the iron oxide particles are comprised in the composite material in a weight ratio of 0.001 to 0.200 with respect to the ZnO support particles. In a more particular embodiment, the iron oxide is FeOOH, preferably in a weight ratio of 0.02 to 0.10 with respect to the ZnO support particles. In another more particular embodiment, the iron oxide is Fe₂O₃, preferably in a weight ratio of 0.003 to 0.030 the ZnO support particles. In a further more particular embodiment, the iron oxide is Fe₃O₄, preferably in a weight ratio of 0.001 to 0.020 to the ZnO support particles. In a further particular embodiment, the iron oxide is a combination of FeOOH, Fe₂O₃ and/or Fe₃O₄, any of them in the weight ratios with respect to the ZnO support particles defined hereinabove.

In a particular embodiment, the composite material comprises at least one further inorganic cosmetic ingredient.

Preferably, the inorganic cosmetic ingredient is selected from the group consisting of phosphates such as hydroxyapatite and other calcium phosphates or potassium phosphates, silicates such as mica or talc; clays such as kaolinite, bentonite or smectite and combinations thereof.

More preferably, the inorganic cosmetic ingredient is selected from the group consisting of kaolin, mica and hydroxyapatite.

In a preferred embodiment, any of these inorganic cosmetic ingredients is present in the amounts that follow below.

In a more particular embodiment, the above further organic and/or inorganic cosmetic ingredients may be added in an amount of 0.5% to 35%, preferably in an amount of 1% to 30% by weight with respect to the total weight of the composite material.

In a particular embodiment, any of these inorganic cosmetic ingredients is comprised in an amount from about 5 to about 25%, preferably from about 8% to about 20% by weight with respect of the total weight of the composite material.

The inorganic cosmetic ingredients of the present invention are typically in the form of finely divided particles. In a particular embodiment, the particles of the inorganic cosmetic ingredient in any of the embodiments described herein, have a mean size of less than 2 microns to 120 microns, preferably a mean size of between 2 to 20 microns. The particle size may be determined by high-resolution electron microscopy or laser diffraction.

In a particular embodiment, the composite material comprises at least one further cosmetic organic ingredient.

Preferably, the organic compound is selected from the group consisting of vitamins, antioxidants and combinations thereof.

Non-limiting examples of vitamins include the B vitamins (e.g., B1, B2, B6, B12, niacin, niacinamide, folic acid, biotin, and pantothenic acid), vitamin C, vitamin D, vitamin E (e.g., tocopherol or tocopheryl acetate), vitamin A (e.g., retinyl palmitate, or retinoic acid), and vitamin K. Non-limiting examples of antioxidants include argan (Argania spinosa leaf extract), beta-carotene (carrot extract), butylated hydroxyanisole (BHA) and butylated hydroxytoluene (BHT).

In a preferred embodiment, the organic cosmetic ingredient is a vitamin selected from the group consisting of A vitamins, B vitamins, E vitamins and combinations thereof. In another preferred embodiment, the organic cosmetic ingredient is an antioxidant selected from the group consisting Argania spinosa leaf extract, carrot extract and combinations thereof.

More preferably, the organic cosmetic ingredient is selected from the group consisting of niacinamide, Argania spinosa leaf extract, carrot extract and combinations thereof.

In a preferred embodiment, any of these organic cosmetic ingredients is present in the amounts that follow below.

In a particular embodiment, any of these organic cosmetic ingredients is comprised in an amount from about 0.5 to about 35%, preferably from about 1% to about 30% by weight with respect of the total weight of the composite material.

In a preferred embodiment, the organic cosmetic ingredient is niacinamide in a 20% to 30% by weight with respect to the total weight of the composite material. In another preferred embodiment, the organic cosmetic ingredient is Argania spinosa leaf extract in a 1% to 2% by weight with respect to the total weight of the composite material. In a further preferred embodiment, the organic cosmetic ingredient is carrot extract in a 15% to 20% by weight with respect to the total weight of the composite material.

The organic cosmetic ingredients of the present invention are typically in the form of finely divided particles. In a particular embodiment, the particles of the organic cosmetic ingredient in any of the embodiments described herein, have a mean size of 50 nm to 500 microns, preferably a mean size of 50 to 300 microns.

In a particular embodiment, the composite material of the present invention has a relative particle size distribution D50 between 1 and 10 microns, preferably between 2 and 7 microns. D50 refers to the particle size corresponding to a cumulative particle size distribution percentage of 50%, also called median particle size. Said particle size distribution can be measured by laser diffraction, such as by using a laser diffraction particle size analyser such as a Malvern Mastersizer 2000 instrument, as described in detail in Example 1 below.

In a preferred embodiment, the composite material of the present invention comprises:
ZnO support particles, as in any of the embodiments described above, in a 65 to 83%, preferably in a 70 to 80% by weight, and
ZnO nanoparticles as in any of the embodiments described above, in a 17 to 35%, preferably in a 20 to 30% by weight,
wherein said amounts are expressed with respect to the total weight of the composite material.

In a further preferred embodiment, the composite material of the present invention comprises:
ZnO support particles, as in any of the embodiments described above,
ZnO nanoparticles, as in any of the embodiments described above, in a weight ratio from about 0.2 to about 0.6, preferably from about 0.25 to about 0.45 with respect to the ZnO support particles, and
iron oxide particles, as in any of the embodiments described above, in a 1 to 10% by weight with respect to the total weight of the composite material.

In a further preferred embodiment, the composite material of the present invention comprises:
ZnO support particles, as in any of the embodiments described above,
ZnO nanoparticles, as in any of the embodiments described above, in a weight ratio from about 0.2 to about 0.6, preferably from about 0.25 to about 0.45 with respect to the ZnO support particles, and
a further cosmetic ingredient, preferably selected from niacinamide, Argania spinosa leaf extract or carrot extract, in a 0.5 to 35% by weight with respect to the total weight of the composite material.

In another preferred embodiment, the composite material of the present invention consists of:
ZnO support particles, as in any of the embodiments described above, in a 65 to 83%, preferably in a 70-80% by weight, and
ZnO nanoparticles, as in any of the embodiments described above, in a 17 to 35%, preferably in a 20-30% by weight,
wherein said amounts are expressed with respect to the total weight of the composite material.

As mentioned above, the composite materials of the present invention provide for high sun protection factor (SPF) values and excellent stability upon incorporation to a cosmetic sunscreen formulation.

Thus, another aspect of the invention relates to a cosmetic composition comprising the composite material of the present invention in any of its particular embodiments.

The cosmetic compositions of the present invention have UV-protective properties attributable to the incorporated composite material. The ultraviolet (“UV” herein) region of the electromagnetic spectrum comprises three wavebands, designated UVA from 320 to 400 nm, UVB from 290 to 320 nm and UVC from 200 to 290 nm. The visible region of the spectrum is generally from about 380 to about 780 nm.

Preferably, the cosmetic composition is a sunscreen composition.

Sunscreen compositions typically are a topical formulation for application to the skin that helps protect against sunburn and related damage to the skin. Sunscreens may be in the form of lotions, sprays, gels, foams, sticks, powders and other topical products.

In a particular embodiment, the sunscreen composition of the present invention is an oil-in-water emulsion or a water-in-oil emulsion comprising at least the following components:
(a) the composite material of the present invention in any of the embodiments described above,
(b) an emulsifier,
(c) an aqueous component, and
(d) an oil component.

The chosen emulsifier, depending upon its chemical nature, will be a component of either the oil or aqueous phase, and assists with both the formation and the maintenance, or stability, of the emulsion. Preferably, the emulsifier is a component of the oil phase.

Mostly non-ionic emulsifiers for sunscreen and other cosmetic formulations can be used in the invention. Non-limiting examples of emulsifiers include Arachidyl Alcohol, Behenyl Alcohol, Arachidyl Glucoside, Cetearyl Alcohol, Cetearyl Glucoside, Cetearyl Olivate, coco-glucoside, Coconut Alcohol, C14-22 Alcohols (and) C12-20 Alkyl Glucoside, Glyceryl Stearate, Olive Oil Polyglyceryl-6 Esters, Polyhydroxystearic Acid, Polyglyceryl-2 Stearate, Polyglyceryl-3 Diisostearate, Polyglyceryl-2 dipolyhydroxystearate, Polyglyceryl-6 Polyhydroxystearate, Polyglyceryl-3 Polyricinoleate, Polyglyceryl-6 Pentaoleate, Polyglyceryl-6 Polyricinoleate Sorbitan Olivate, Stearyl Alcohol.

In a particular embodiment, the emulsifier is selected from Cetearyl alcohol (and) cocoglucoside, C14-22 Alcohols (and) C12-20 Alkyl Glucoside, and combinations thereof.

In a particular embodiment, the amount of emulsifier used in sunscreen formulation is of 1 to 10% by weight with respect to the total weight of the formulation.

The oil component comprises a mixture of oils and/or fats. Most of the widely used oils and fats for sunscreen formulations can be used in the invention. Non-limiting examples include Butylene Glycol Cocoate, Butyloctyl Salicylate, Caprylic/Capric Triglyceride, Coco-Caprylate/Caprate, Cocoglycerides, Cocos Nucifera (Coconut) Oil, Aloe, Dibutyl Adipate, Hydrogenated Castor Oil, Hydrogenated Ethylhexyl Olivate (and) Hydrogenated Olive Oil Unsaponifiables, Hydrogenated Vegetable Oil, Isoamyl Cocoate, Isoamyl Laurate, Jojoba Esters (and) Helianthus Annuus (Sunflower) Seed Wax (and) Acacia Decurrens Flower Wax (and) Polyglycerin-4, Lauryl Olivate, Neopentyl Glycol Diethylhexanoate, Neopentyl Glycol Diheptanoate, Polyhydroxystearic Acid (and) Neopentyl Glycol Diethylhexanoate, Prunus Amygdalus Dulcis Oil, Simmondsia Chinensis (Jojoba) Seed Oil, Tridecyl Trimellitate, Triheptanoin, Triisostearin, Vegetable Oil (and) Simmondsia Chinensis (Jojoba) Seed Oil (and) Curcuma Longa (Turmeric) Root Extract, Tricaprylin, Wax, and combinations thereof.

The aqueous component comprises water and optionally other water-soluble ingredients of the formulation.

Advantageously, the composite material of the present invention in any of the embodiments described above may be easily dispersed either in the aqueous or the oil phase of a sunscreen emulsion and provides excellent stability without observing increase of pH or phase migration phenomena over time; effects that usually lead to poor stability of the emulsion and phase separation.

Thus, in some embodiments the composite material of the invention may be incorporated to the aqueous component of the present invention.

When the composite material is present in the aqueous component, said aqueous component preferably comprises a viscosity modifier. As used herein, the term viscosity modifier refers to an excipient that increases the viscosity of the aqueous component. The viscosity modifier is preferably a gum, more preferably selected from the group consisting of lecithin, lysolecithin, Dehydroxanthan Gum, xantham gum, cellulose, microcrystalline cellulose, pullulan, sclerotium gum, Chondrus crispus Powder and combinations thereof. In such cases, the viscosity modifier is preferably in an amount between 0.1 and 5.0% by weight, relative to the total weight of the sunscreen formulation, more preferably in an amount between 0.1 and 2.5% relative to the total weight of the sunscreen formulation. In a preferred embodiment, the viscosity modifier is xanthan gum, preferably in an amount between 0.3 and 0.5% by weight relative to the total weight of the formulation. In another preferred embodiment, the viscosity modifier is a combination of xanthan gum and microcrystalline cellulose in an amount of about 2% by weight relative to the total weight of the formulation.

In some alternative embodiments, the composite material of the present invention is incorporated to the oil component of the emulsion, preferably a water-in-oil emulsion. When the composite material is present in the oil component, said oil component preferably comprises a dispersant. As used herein, the term dispersant refers to an excipient that helps preventing agglomeration of the composite material. In preferred embodiments, the dispersant is selected from the group consisting of butyloctyl salicylate, Coco-Caprylate/Caprate, Cocoglycerides, Isoamyl Cocoate, Neopentyl Glycol Diethylhexanoate, Neopentyl Glycol Diheptanoate, Polyhydroxystearic Acid (and) Neopentyl Glycol Diethylhexanoate, Tridecyl Trimellitate Triisostearin an oil or a wax In such cases, the dispersant or dispersant combination is preferably in an amount, depending on the composite concentration, between 5 and 25% by weight, preferably between 10 and 20% by weight relative to the total weight of the sunscreen formulation.

The sunscreen formulation may comprise further ingredients commonly used in sunscreen formulations such as emollients, film-formers, humectants, oils, waxes, preservatives, fragrances.

The sunscreen emulsions of the present invention may be obtained by common methods known in the art. For example, the ingredients of the oil component are melted under stirring and added to the aqueous component, previously heated to the same temperature of the oil component, under stirring to emulsify and the emulsion subject to high pressure mixing such as homogenization.

It has been surprisingly found that sunscreen formulations containing the composite materials of the present invention as sunscreen active agents show unexpectedly high sun protection properties for a ZnO based sunscreen agent.

The effectiveness of a sunscreen product is indicated by its sun protection factor (SPF). The sun protection factor is a measure of the solar energy required to produce sunburn on protected skin (i.e., in the presence of sunscreen) relative to the amount of solar energy required to produce sunburn on unprotected skin. For example, “SPF15 means that it takes 15 times longer to induce erythema in treated skin compared to unprotected skin, assuming sunscreen is applied evenly at a thick dosage of 2 milligrams per square centimeter (mg/cm2).

SPF may be measured in vitro by a spectrophotometer. The test is based on the evaluation of UV transmittance through a thin layer of sunscreen (1.3 mg/cm²) distributed on a substrate of controlled roughness. SPF and UVA are calculated from the transmittance spectra at wavelengths 290-400 nm. Sun protection product transmission data are mathematically adjusted, so that the in vitro SPF data give values similar to SPF results obtained through in vivo testing.

In a particular embodiment, the cosmetic composition has a Sun Protection Factor (SPF) of at least 15, at least 20, at least 25, at least 30, at least 35, at least 40, at least 50, at least 60, at least 70. In a particular embodiment, the cosmetic composition has a Sun Protection Factor (SPF) of at about 30, about 35, about 40, about 50, about 60, about 70 or about 80.

In a particular embodiment, the sunscreen formulation comprising the composite material of the present invention has a UVA/UVB ratio of 0.6 to 0.9, preferably 0.65 to 0.80.

The UVA/UVB ratio is a relative measure of the UVB versus UVA absorbance of the sunscreen formulation. The absorbance of a thin film of the product is summed for the UVB and UVA ranges and the UVA/UVB absorbance ratio is calculated to classify products into five classes of protection level, where the closer the ratio is towards unity, the higher the rating of the product. For example, a UVA/UVB ratio of greater than 0.91 is labeled as “Ultra” or “5.”

In a particular embodiment, the sunscreen formulation comprising the composite material of the present invention has a critical wavelength of 370 or longer.

Critical Wavelength is used to measure the “breadth” of UV protection. In particular, it can be defined as the wavelength at which the summed absorbance reaches 90% of total absorbance. This parameter can be determined by methods well-known in the art. Typically, the absorbance of a thin film of the sunscreen is integrated from 290 nm across the UV spectrum until the sum reaches 90% of the total absorbance of the sunscreen in the ultraviolet region (290-400 nm). In one embodiment, a sunscreen formulation may be classified as ‘broad spectrum’ if the critical wavelength is longer than 370 nm.

In a particular embodiment, the concentration of composite material in the cosmetic composition, preferably sunscreen formulation, is between 5% and 25% by weight, preferably between 10% and 20%, more preferably between 15% and 18% by weight with respect to the total weight of the composition.

In some embodiments, when a composite material which further comprises a pigment is used, the cosmetic composition is preferably a makeup foundation. It provides a strong moisturizing and freshness sensation, creamy texture with very comfortable feeling during application, and sheer natural makeup result after application. Such composition provides makeup efficacy (proper coverage and natural radiance) in addition to a sunscreen effect.

In another aspect, the present invention relates to a method for the preparation of the composite material of the present invention, said method comprising the steps of:
(i) providing ZnO support particles with a mean size between 120 and 800 nm,
(ii) adding ZnO nanoparticles with a mean size of less than 100 nm to said support particles, and
(iii) stirring in a dry medium to disperse the nanoparticles onto the support particles at a speed from 5 to 100 rpm.

Particular and preferred embodiments for the ZnO support particles of step (i) and the ZnO nanoparticles of step (ii) are as those described hereinabove.

In a particular embodiment, the ZnO nanoparticles of step (ii) are added in a weight ratio of 17% to 35% with respect to the total weight of the ZnO support particles provided in step (i). Preferably the ZnO nanoparticles of step (ii) are added in a weight ratio of 15% to 37%, preferably of 19% to 30%, more preferably 20% to 28% with respect to the total weight of ZnO particles, i.e., ZnO support particles and ZnO nanoparticles.

In a particular embodiment, step (ii) comprises adding at least one further ingredient, in particular at least one further cosmetic ingredient to the support particles.

In a particular embodiment, the further cosmetic ingredient added in step (ii) is a pigment, as in any of the embodiments described above. In another particular embodiment, the further cosmetic ingredient is an organic and/or an inorganic cosmetic ingredient, as in any of the embodiments described above.

Preferably, the inorganic cosmetic ingredient is selected from the group consisting of phosphates, silicates; clays and combinations thereof. More preferably, the inorganic cosmetic ingredient is selected from the group consisting of kaolin, mica and hydroxyapatite.

Preferably, the organic cosmetic ingredient is a vitamin selected from A vitamins, B vitamins, E vitamins; an antioxidant selected from Argania spinosa leaf extract, carrot extract; or combinations thereof.

In a particular embodiment, the further cosmetic ingredient added in step (ii) is iron oxide, such as in the form of particles. Particular and preferred embodiments for the iron oxide particles added in step (ii) are as those described above.

In a more particular embodiment, the iron oxide particles are added in an amount from about 0.1 to about 15%, preferably from about 1% to about 10%, more preferably from about 2% to 8% by weight with respect of the total weight of the composite material.

In a more particular embodiment, the iron oxide is FeOOH, preferably added in an amount from about 2% to 8% by weight with respect of the total weight of the composite material. In another more particular embodiment, the iron oxide is Fe₂O₃, preferably added in an amount from about 0.2% to about 2% by weight with respect of the total weight of the composite material. In another more particular embodiment, the iron oxide is Fe₃O₄, preferably added in an amount from about 0.1% to about 1% by weight with respect of the total weight of the composite material.

In another particular embodiment, the iron oxide is a combination of FeOOH, Fe₂O₃, and/or Fe₃O₄, each of them added in any of the amounts described hereinabove.

In another particular embodiment, the iron oxide particles are added in a weight ratio of 0.001 to 0.200 with respect to the ZnO support particles.

In a particular embodiment, the iron oxide particles may be selected from FeOOH, Fe₂O₃, Fe₃O₄ and combinations thereof. In a more particular embodiment, the iron oxide is FeOOH, preferably in a weight ratio of 0.02 to 0.10 with respect to the ZnO support particles. In another more particular embodiment, the iron oxide is Fe₂O₃, preferably in a weight ratio of 0.003 to 0.030 the ZnO support particles. In a further more particular embodiment, the iron oxide is Fe₃O₄, preferably in a weight ratio of 0.001 to 0.020 to the ZnO support particles.

In another particular embodiment, the iron oxide particles are a combination of FeOOH, Fe₂O₃, and/or Fe₃O₄, each of them in the weight ratios with respect to the ZnO support particles described hereinabove.

In another particular embodiment, the further cosmetic ingredient added in step (ii) is selected from the group consisting of niacinamide, Argania spinosa extract, carrot extract, kaolin, mica hydroxyapatite and combinations thereof; which are known for their anti-inflammatory properties.

In a more particular embodiment, the above further organic and/or inorganic cosmetic ingredients may be added in an amount of 0.5% to 35%, preferably in an amount of 1% to 30% by weight with respect to the total weight of the composite material.

The stirring of step (iii) is performed in a dry medium. As used herein, the term “dry medium” refers to the absence of any liquid solvent. The method of this invention uses low shear mixing for the dry dispersion of nanoparticles, which allows obtaining a new class of materials in which ZnO nanoparticles are dispersed on larger ZnO support particles.

Collisions between the agglomerates of particles and nanoparticles generated in low speed regimes cause breakage of said agglomerates and as a result the nanoparticles are dispersed on the surface of the support particles giving rise to a new kind of material with a hierarchical structure the nanoparticles are supported on the outer surface of the support particles.

The stirring of step (iii) is performed at a speed of 5 to 100 rpm. Alternatively, the stirring speed may be expressed as the angular velocity in rad/s, being from 0.5 to 10.5 rad/s, or the stirring speed may be expressed as the rotation speed in Hertz, being from 0.08 to 1.7 Hz.

In a particular embodiment, the stirring of step (iii) is performed at a speed of 7 to 70 rpm, more preferably 10 to 60 rpm, even more preferably between 12 and 40 rpm. Alternatively, the stirring speed may be expressed as the angular velocity in rad/s, being from 0.7 to 7.3 rad/s, more preferably 1 to 6.3 rad/s, even more preferably between 1.25 and 4.2 rad/s or the stirring speed may be expressed as the rotation speed in Hertz, being from 0.1 to 1.17 Hz, more preferably 0.16 to 1 Hz, even more preferably between 0.2 and 0.67 Hz.

The stirring of step (iii) is typically carried out within a mixer where the support particles of step (i) are located and to which at least the ZnO nanoparticles of step (ii) are added. Upon stirring in said mixer, deposition of nanoparticles on the support particles surface occurs. The stirring of step (iii) may be performed for example, without limitation, in a shaker mixer, such as a double cone mixer, drum mixer, V mixer, Turbula type mixer, free-fall mixer, planetary mixer, intensive Eirich mixer. Preferably, the stirring is performed in a double cone mixer. The mixer may be partially charged with particles and nanoparticles to be dispersed so that collisions between the agglomerates of different powder materials are favored during mixing.

The dispersion process can be extended to include several types of cosmetic ingredients either simultaneously with the dispersion of ZnO nanoparticles or sequentially after the preparation of a first hierarchical ZnO composite material by dispersing one or more further ingredients on said composite material.

In a particular embodiment, the method further comprises the steps of:
(iv) adding at least one further ingredient to the composite material of step (iii), and
(v) stirring in a dry medium to disperse said ingredient onto the support particles of the composite material at a speed from 5 to 100 rpm.

In a particular embodiment, the further cosmetic ingredient added in step (iv) is a pigment, as in any of the embodiments described above. In another particular embodiment, the further cosmetic ingredient is an organic and/or an inorganic cosmetic ingredient, as in any of the embodiments described above.

Preferably, the inorganic cosmetic ingredient is selected from the group consisting of phosphates, silicates; clays and combinations thereof. More preferably, the inorganic cosmetic ingredient is selected from the group consisting of kaolin, mica and hydroxyapatite.

Preferably, the organic cosmetic ingredient is a vitamin selected from A vitamins, B vitamins, E vitamins; an antioxidant selected from Argania spinosa leaf extract, carrot extract; or combinations thereof.

In a particular embodiment, any of these any of these further cosmetic ingredients may be added in an amount of 0.5% to 35%, preferably 1% to 30% by weight, with respect to the total weight of the composite material.

In a preferred embodiment, the further cosmetic ingredient added in step (iv) is selected from the group consisting of niacinamide, Argania spinosa leaf extract, carrot extract, kaolin, mica hydroxyapatite and combinations thereof.

In a more preferred embodiment, the further cosmetic ingredient added in step (iv) is niacinamide in a 20% to 30% by weight with respect to the total weight of the composite material. In another more preferred embodiment, the further cosmetic ingredient added in step (iv) is Argania spinosa leaf extract in a 1% to 2% by weight with respect to the total weight of the composite material. In a further more preferred embodiment, the further cosmetic ingredient added in step (iv) is carrot extract in a 15% to 20% by weight with respect to the total weight of the composite material.

In another particular embodiment, the further cosmetic ingredient added in step (iv) is iron oxide, such as in the form of particles. Particular and preferred embodiments for the iron oxide particles added in step (ii) are as those described above.

In a more particular embodiment, the iron oxide particles are added in a 0.1% to 10%, preferably 1 to 8% by weight with respect of the total weight of the composite material.

The stirring of step (v) is performed in a dry medium, i.e., in the absence of solvent.

The stirring of step (v) is performed at a speed of 5 to 100 rpm. Alternatively, the stirring speed may be expressed as the angular velocity in rad/s, being from 0.5 to 10.5 rad/s, or the stirring speed may be expressed as the rotation speed in Hertz, being from 0.08 to 1.7 Hz.

In a particular embodiment, the stirring of step (v) is performed at a speed of 7 to 70 rpm, more preferably of 10 to 60 rpm, even more preferably between 12 and 40 rpm. Alternatively, the stirring speed may be expressed as the angular velocity in rad/s, being from 0.7 to 7.3 rad/s, more preferably 1 to 6.3 rad/s, even more preferably between 1.25 and 4.2 rad/s or the stirring speed may be expressed as the rotation speed in Hertz, being from 0.1 to 1.17 Hz, more preferably 0.16 to 1 Hz, even more preferably between 0.2 and 0.67 Hz.

In a particular embodiment, the stirring of step (v) is performed in a shaker mixer, preferably selected from the group consisting of a double cone mixer, drum mixer, V mixer, Turbula type mixer, free-fall mixer, planetary mixer and intensive Eirich mixer. More preferably, the stirring of step (v) is performed in a double cone mixer. The mixer may be partially charged with the material composite of step (iii) and further ingredient(s) to be dispersed so that collisions between the agglomerates of different powder materials are favored during mixing.

In a preferred embodiment, the method comprises:
(i) providing ZnO support particles, as in any of the embodiments described above,
(ii) adding to said support particles ZnO nanoparticles, as in any of the embodiments described above, preferably in a weight ratio of 0.25 to 0.45 with respect to the ZnO support particles, and iron oxide particles, preferably in a weight ratio of 0.001 to 0.200 with respect to the ZnO support particles,
(iii) stirring in a dry medium, preferably at a speed of 10 to 60 rpm, to disperse the ZnO nanoparticles and iron oxide particles onto the support particles.

Within this particular embodiment, the iron oxide particles may be selected from FeOOH, Fe₂O₃, Fe₃O₄ and combinations thereof. In a more particular embodiment, the iron oxide is FeOOH, preferably in a weight ratio of 0.02 to 0.1 with respect to the ZnO support particles. In another more particular embodiment, the iron oxide is Fe₂O₃, preferably in a weight ratio of 0.003 to 0.03 the ZnO support particles. In a further more particular embodiment, the iron oxide is Fe₃O₄, preferably in a weight ratio of 0.001 to 0.02 to the ZnO support particles.

In a further preferred embodiment, the method of the present invention comprises:
(i) providing ZnO support particles, as in any of the embodiments described above,
(ii) adding ZnO nanoparticles, as in any of the embodiments described above, to said support particles, preferably in a weight ratio of 0.25 to 0.45 to the ZnO particles, and
(iii) stirring in a dry medium, preferably at a speed between 10 to 60 rpm, to disperse the nanoparticles onto the support particles.

For this particular embodiment, the method may further comprise:
(iv) adding a further cosmetic ingredient to the composite material obtained in step (iii), preferably an organic ingredient selected from the group consisting of vitamins, antioxidants, and combinations thereof, and/or an inorganic ingredient selected from phosphates, silicates, clays and combinations thereof.
(v) stirring in a dry medium, preferably at a speed between 10 to 60 rpm, to disperse said ingredient onto the support particles of the composite material.

Within this particular embodiment, particular and preferred embodiments for the organic and inorganic cosmetic ingredients are as those defined above. More preferably, the further cosmetic ingredient is selected from niacinamide, Argania spinosa extract, carrot extract, kaolin, mica, hydroxyapatite and combinations thereof.

The method of the present invention allows the dry dispersion of ZnO nanoparticles on other ZnO bigger support particles acting as support using low shear mixing speeds, which allows obtaining a new class of ZnO hierarchical materials active as sunscreen agents.

Accordingly, in another aspect the present invention refers to a hierarchical composite material obtainable by the method as described above, in any of its particular embodiments.

In a further aspect relates to a method of filtering UVA and UVB radiation, said method comprising the topical application of the composite material according to the present invention in any of its particular embodiments, or the cosmetic formulation according to the present invention in any of its particular embodiments to a subject in need thereof.

In a sixth aspect, the invention relates to a method of treatment or prevention of skin inflammation, said method comprising administering an effective amount of the hierarchical composite material of the invention, or of the hierarchical composite material of the invention further comprising at least one organic cosmetic ingredient selected from the group consisting of niacinamide, Argania spinosa leaf extract, carrot extract and combinations thereof to a subject in need thereof.

According to a preferred embodiment said administration is performed by topical application of the hierarchical composite material of the invention or of the hierarchical composite material of the invention further comprising at least one organic cosmetic ingredient selected from the group consisting of niacinamide, Argania spinosa leaf extract, carrot extract and combinations thereof.

Unless defined otherwise, all technical and scientific terms used herein have the same meaning as commonly understood to one of ordinary skill in the art to which this disclosure belongs. As used herein, the singular forms “a”, “an” and “the” include plural reference unless the context clearly dictates otherwise.

As used herein, the terms “about” or “around” mean a slight variation of the value specified, preferably within 10 percent of the value specified. Nevertheless, the term “about” or the term “around” can mean a higher tolerance of variation depending on for instance the experimental technique used. Said variations of a specified value are understood by the skilled person and are within the context of the present invention.

All the features described in this specification (including the claims, description and drawings) and/or all the steps of the described method can be combined in any possible combination, with the exception of combinations of such mutually exclusive features and/or steps.

### EXAMPLES

The invention is illustrated by means of the following examples which in no case limit the scope of the invention.

### Example 1: Preparation of Zn/Zn Composite Materials

360 g of ZnO support particles with an average particle size of 150-600 nm and 120 grams of ZnO nanoparticles with an average size of 10-90 nm were used. The two ceramic powders were added to the mixing chamber of a bicone or two-cone mixer of 5 L lab scale mixer, material: AISI 316 L stainless steel, In contact with the product polishing Ra<0.6 μm, exterior polished Ra<0.8 μm. The mixture was stirred at a speed of 25 rpm for 30-120 minutes.

Several composite materials were prepared by varying the percentage of nanoparticles from 17 to 30% by weight, and the percentage of support particles from 50 to 83% by weight.

Particle size distribution and Z average of the composite materials was evaluated by laser diffraction using a Malvern Mastersizer 2000 instrument equipped with the measurement accessory (Scirocco) using 2 bar of pressure to disperse the solid sample. Procedure: The measurements are made in triplicate, measuring in each of them 10 seconds of background and 10 seconds of data collection with the sample circulating. The sample addition is adjusted so that the darkening is between 1-3%. In the measurement of solids, a red laser is used. The processing of the experimental data was carried out using the MIE theory.

Nitrogen adsorption/desorption isotherms were recorded on a Micromeritics ASAP-2000 device. Before performing the analysis, the samples were degassed for 15 hours at 120° C. and 10\hat{ }-6 Torr. The surface of the samples was estimated using the BET model

Zeta Potential measurements were carried out in a Malvern Zetasizer Nano ZS, using a DTS1070 cuvette. The measurement was performed in triplicate, accumulating between 10-15 cycles per measurement.

The different results for the corresponding ZnZn composite materials studied are summarized in Table 2.

TABLE 1
Characterization of ZnZn materials.

| | Particle size | Specific surface | Z potential ± |
|---|---|---|---|
| | D(v, 0.5) | area BET | std |
| Material | (μm) | (m²/g) | (mV) |
| ZnZn-17% NP | 5.584 | 6.5995 ± 0.0799 | −8.72 ± 9.8 |
| ZnZn-20% NP | 5.001 | N/A* | 2.93 ± 15.0 |
| ZnZn-22.5% NP | 5.974 | N/A | 5.97 ± 15.3 |
| ZnZn-25% NP | 3.127 | 11.8694 ± 0.0264 | 21.5 ± 4.2 |
| ZnZn-30% NP | 3.990 | 19.8550 ± 0,1178 | 21.5 ± 6 |

*N/A = not available

Samples ZnZn-17% NP, ZnZn-20% NP and ZnZn-22.5% NP showed higher particle size and lower Z potential due to higher proportion of support particles of higher size and negative surface charge values, whereas ZnZn-25% and NPZnZn-30% NP exhibited lower particle size and clear positive Z potential due to higher proportion of nanoparticles and positive surface charges. Specific surface area BET increases with the nanoparticles content.

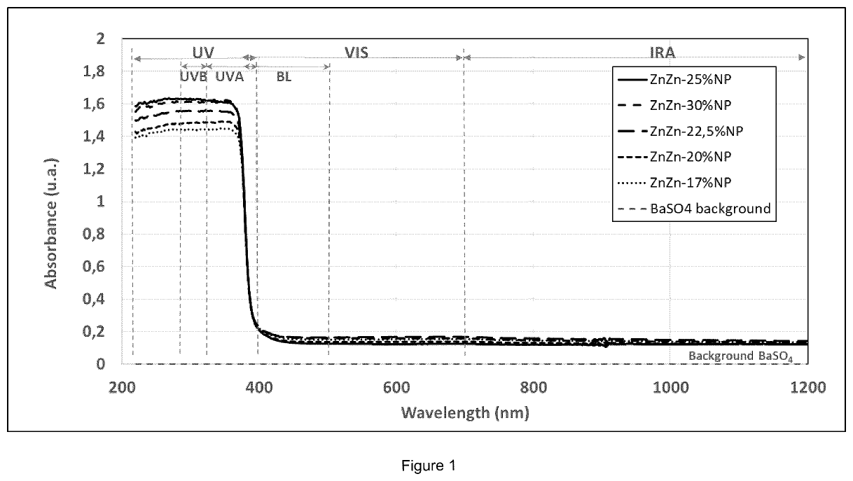

The UV-Vis-IR absorbance behavior of these materials was measured by transmittance in a Shimadzu UV-2600i UV-Vis-NIR spectrophotometer instrument with Integrating Sphere Attachment (solid samples). The measurement shows the absorption of radiation at different wavelengths (200-1200 nm) and therefore the protection of these UV filters from radiation from different components of the spectrum (FIG. 1).

For a more sensitive comparison, these materials were dispersed in the aqueous phase of a sunscreen cosmetic formulation with 15 wt % ZnO (INCI: Aqua, Zinc Oxide, Butylene Glycol Cocoate, Butyloctyl Salicylate, Cetearyl alcohol & Cocoglucoside, Dimethicone, Butyrospermum Parkii Butter, C14-22 Alcohol & C12-20 Alkyl Glucoside, Microcrystalline Cellulose and Cellulose Gum, Phenoxyethanol, Ethylhexylglycerin; reference named ADP17-TX). The following day (time 1 d), the SPF, UVA PF and critical wavelength (CW) values of the sunscreens were measured in vitro with the SPF-290AS analyzer from Solar Light (a spectrophotometer specifically designed to provide in vitro testing by measuring the transmittance). By following an internal protocol, adapted from ISO 24443, 1.3 mg/cm2 of sunscreen were spread over 50×50 mm2 PMMA plate of 5 microns-controlled roughness (Schönberg Gmbh. & Co, 3 plates, 9 measurements on each plate). The results are shown in Table 2 below.

TABLE 2
SPF data of sunscreen formulation containing ZnZn materials.

| Material | SPF ± std | UVA ± std | UVA/UVB | CW¹ |
|---|---|---|---|---|
| ZnZn-17% NP | 34 ± 8 | 16 ± 3 | 0.65 | 376 |
| ZnZn-20% NP | 44 ± 5 | 20 ± 2 | 0.66 | 376 |
| ZnZn-22.5% NP | 51 ± 5 | 24 ± 3 | 0.66 | 376 |
| ZnZn-25% NP | 53 ± 5 | 25 ± 3 | 0.66 | 376 |
| ZnZn-30% NP | 34 ± 2 | 17 ± 1 | 0.67 | 376 |

¹The critical wavelength (CW) is the wavelength below which 90% of the area under the absorbance curve resides. The critical wavelength must be at 370 nm, or longer to achieve broad-spectrum protection.

Scanning Electron Microscopy (SEM) powder images were obtained with the equipment ELECTRON BEAM LITHOGRAPHY ELINE-PLUS from RAITH GMBH. Powder samples were treated with a sputtering metallizer of Cr, Quorum, Q150T S, to give conductivity (chromium layer of 10 nm).

SEM powder-in-ethanol images were also obtained with the equipment ELECTRON BEAM LITHOGRAPHY ELINE-PLUS from RAITH GMBH. In this case, powder samples were dispersed in ethanol (12-15 mg in 30 μL) by stirring with an IKA lab dancer shaker (2800 rpm); then, an aliquot of the supernatant (with the smallest particles) was taken and dried. Dry samples were treated with a sputtering metallizer of Cr, Quorum, Q150T S, to give conductivity (chromium layer of 10 nm).

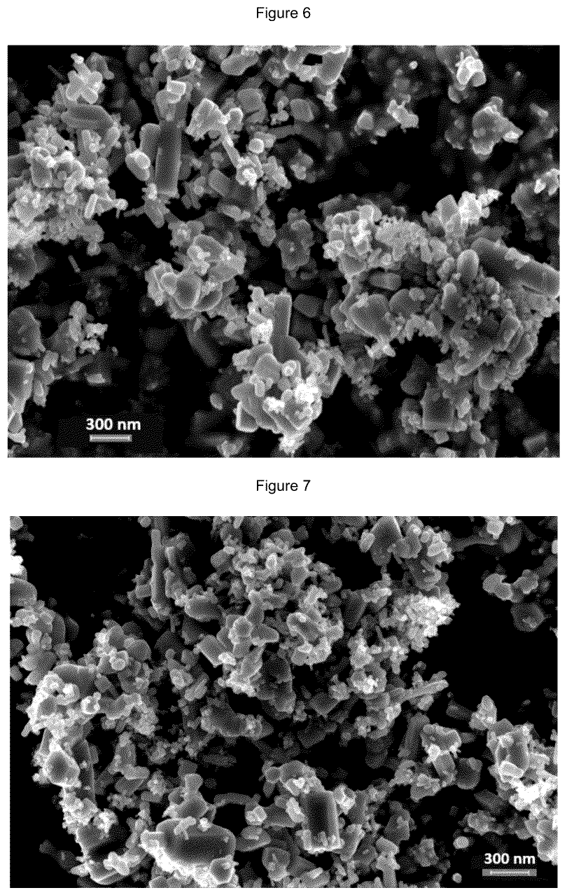

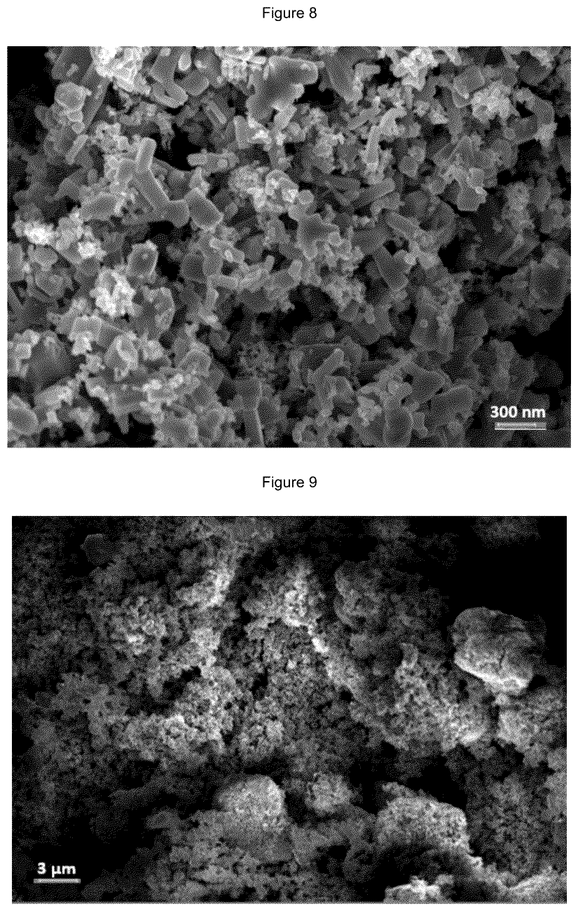

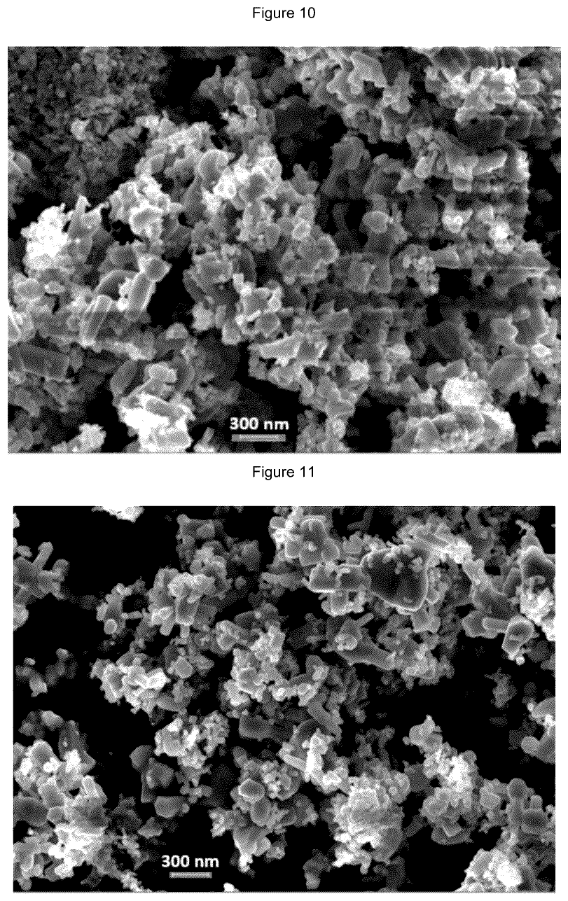

SEM powder images of ZnZn composite materials as described in this example are shown in FIGS. 6-11.

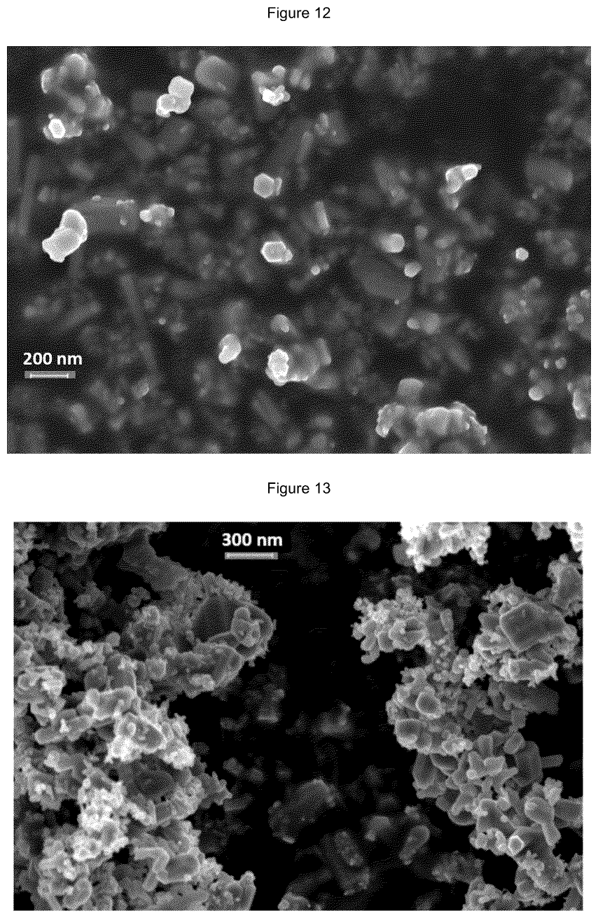

SEM powder-in-ethanol image of ZnZn-25 composite as described in this example is shown in FIG. 12.

### Example 2: ZnZn-Color Composite Materials

ZnO nanoparticles (20-30% wt) and ZnO (60-75% wt) support particles as those described in Example 1 were combined with FeOOH yellow iron oxide CI-77492 (2-8% wt); Fe₂O₃ red iron oxide CI-77491 (0.2-2% wt) or Fe₃O₄ black iron oxide CI-77499 (0.1-1% wt) particles of D50 10-100 nm, 15-100 nm, 0.5-2.0 μm respectively to obtain ZnZn-color materials with three different shades: light, medium and warm. The corresponding mixture was added to the mixing chamber of a bicone mixer as defined in Example 1, and stirred at a speed of 20-30 rpm for 30-120 minutes.

The materials were characterized in terms of particle size by laser diffraction, BET surface area and Z potential as described in Example 1 and the corresponding results are shown in Table 3.

TABLE 3
Characterization of ZnZn-color composite materials.

| | Particle size | Specific surface | Z potential ± |
|---|---|---|---|
| | D(v, 0.5) | area BET | std |
| Material | (μm) | (m²/g) | (mV) |
| ZnZn-light | 4.5 | N/A | −7.47 ± 5.9 |
| ZnZn-medium | 4.1 | 10.9964 ± 0.0584 | +17.3 ± 3.9 |
| ZnZn-warm | 5.3 | N/A | +21.6 ± 4.2 |

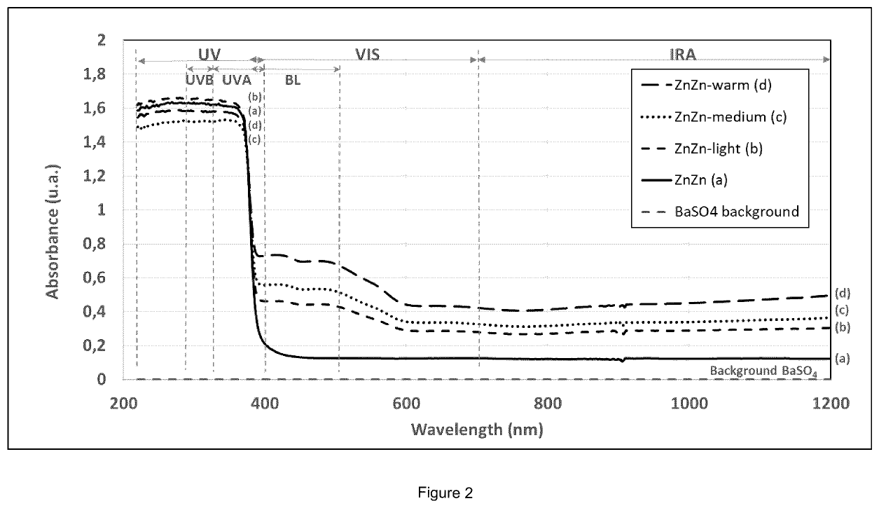

The absorbance of these materials was analyzed in the UV, Vis and near IR (IR-A) regions with a Shimadzu UV-2600i UV-Vis spectrophotometer with integrating sphere (powder samples), as described in Example 1. The results are shown in FIG. 2): A significant protection against UVB and UVA radiation is observed (wavelengths 290-320 and 320-400 nm respectively) for all filters. The area under the absorbance curve and thus the protection properties from UVA (320-400 nm), Vis (from 380 to 700 nm; including blue light from 380-500 nm) and IR radiation (700-1200 nm) increases with the iron oxides content, from light to warm tonalities. The ZnZn-warm filter showed the highest absorption which is related to its higher iron oxide content.

The SEM powder images were obtained as described in Example 1.

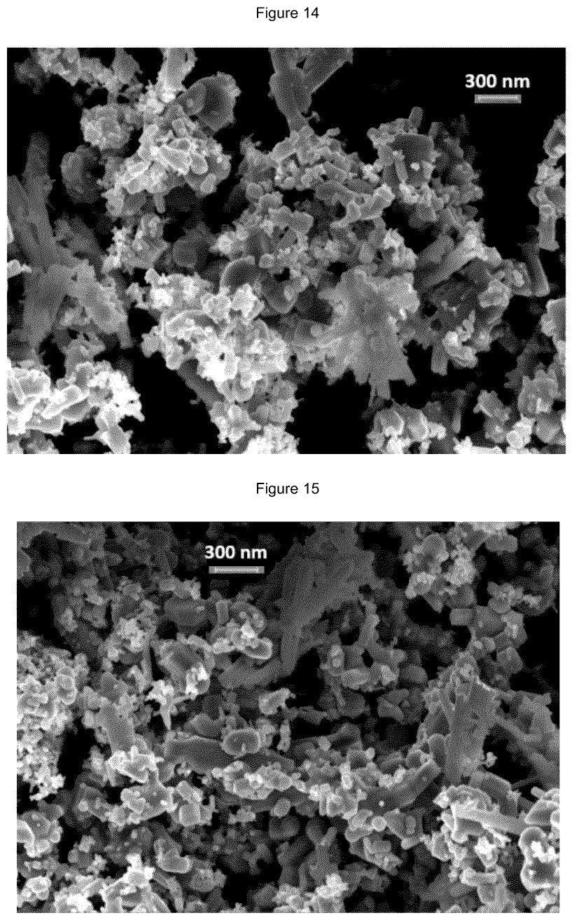

SEM powder images of ZnZn color composite materials ZnZn-light, ZnZn-medium and ZnZn-warm as described in this example are shown in FIGS. 13-15.

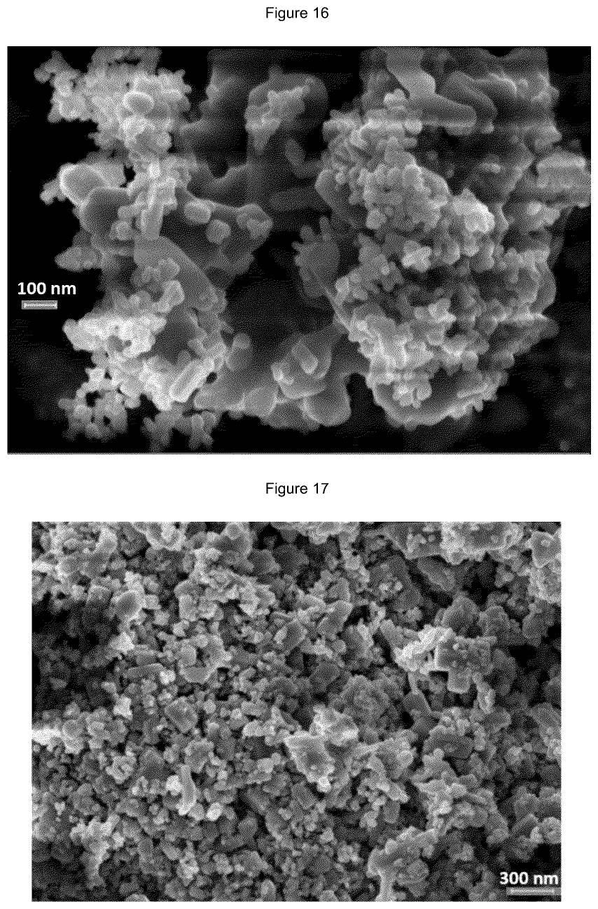

SEM powder-in-ethanol image of ZnZn-warm was obtained as described in Example 1 and is shown in FIG. 16.

### Example 3: ZnZn-Niacinamide Composite

A composite material was prepared by a combination of ZnZn-25 prepared as described in Example 1 (70-80% wt) and niacinamide (20-30% wt powder, purity 100%, CAS No. 98-92-0) as starting materials. The mixture was added to the mixing chamber of a bicone mixer as described in Example 1 and stirred at a speed of 20-30 rpm for 30-120 minutes.

This material was also characterized in terms of particle size by laser diffraction, BET surface area and Z potential as described in Example 1 and the corresponding results are summarized in Table 4.

TABLE 4
Characterization of ZnZn-niacinamide composite.

| | Particle size | Specific surface | Z potential ± |
|---|---|---|---|
| | D(v, 0.5) | area BET | std |
| Material | (μm) | (m²/g) | (mV) |
| ZnZn-niacinamide | 9.3 | 6.8360 ± 0.0875 | 22.1 ± 3.7 |

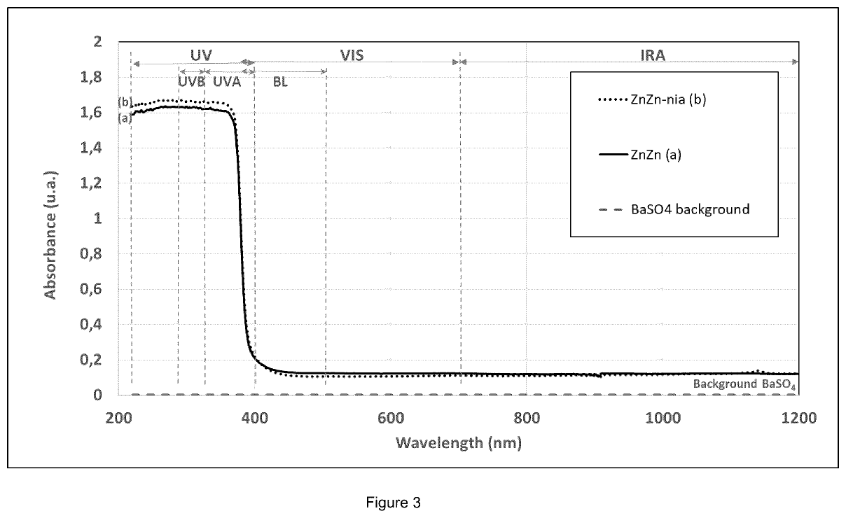

The UV-Vis-IR absorbance was measured as described in Example 1 and the results as compared to ZnZn-25 material are shown in FIG. 3. The absorption of radiation at different wavelengths was similar to that of ZnZn.

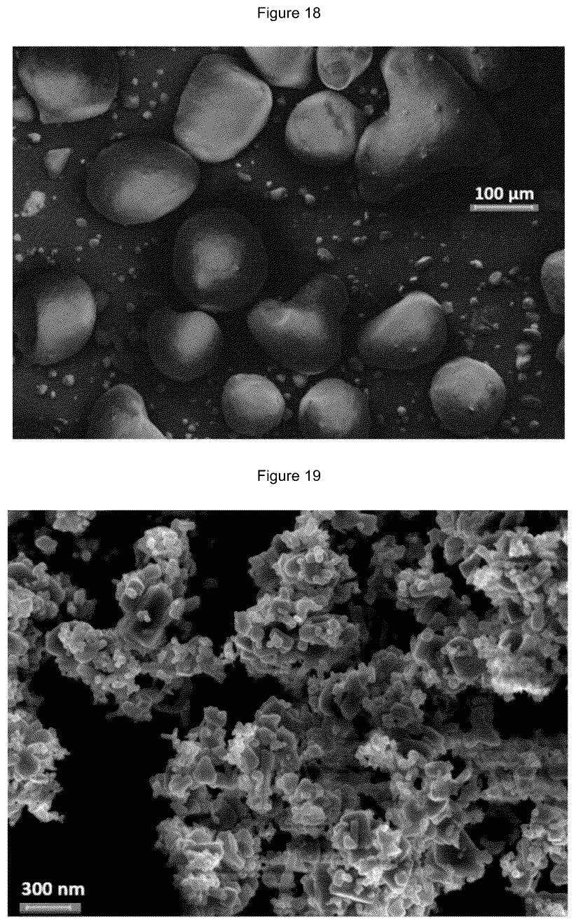

The SEM powder images were obtained as described in Examples 1 and 2. SEM powder images of ZnZn-niacinamide composite as described in this example are shown in FIGS. 17-18.

### Example 4: ZnZn-Carrot Composite

A composite material was prepared by a combination of ZnZn-25 prepared as described in Example 1 (80-85% wt) and carrot extract powder (15-20% wt, Daucus Carota Sativa root powder, orange powder, 77% water soluble, pH 5-6) as starting materials. The mixture was added to the mixing chamber of a bicone mixer as described in Example 1 and stirred at a speed of 20-30 rpm for 30-120 minutes.

This material was also characterized in terms of particle size by laser diffraction, BET surface area and Z potential as described in Example 1 and the corresponding results are summarized in Table 5.

TABLE 5
Characterization of ZnZn-carrot composite.

| | Particle size | Specific surface | Z potential ± |
|---|---|---|---|
| | D(v, 0.5) | area BET | std |
| Material | (μm) | (m²/g) | (mV) |
| ZnZn-carrot | 2.631 | N/A | −18 ± 4.4 |

The SEM powder image was obtained as described in Examples 1-3. SEM powder image of ZnZn-carrot composite as described in this example is shown in FIG. 19.

### Example 5: ZnZn-Argania Composite

Another composite material was prepared by a combination of ZnZn-25 prepared as described in Example 1 (98-99% wt) and Argania Spinosa Leaf Extract (1-2% wt, 70% argania, 30% maltodextrin, yellow powder, pH 4-7 solution 1%) as starting materials. The mixture was added to the mixing chamber of a bicone mixer as described in Example 1 and stirred at a speed of 20-30 rpm for 30-120 minutes.

The results of particle size (laser diffraction), BET surface area and Z potential are shown in Table 6. Results of particle size and BET are similar to those of ZnZn-25% NP while Z potential showed negative values and behavior similar to ZnZn-17% NP.

TABLE 6
Characterization of ZnZn-argania composite.

| | Particle size | Specific surface | Z potential ± |
|---|---|---|---|
| | D(v, 0.5) | area BET | std |
| Material | (μm) | (m²/g) | (mV) |
| ZnZn- | 4.3 | 10.9897 ± 0.0384 | −9.49 ± 7.83 |
| argania | | | |

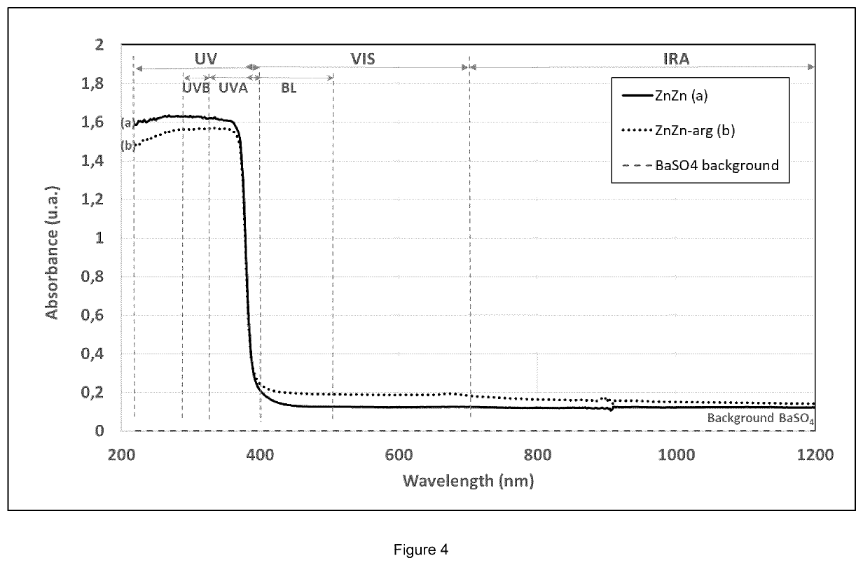

The UV-Vis-IR absorbance obtained with this material was similar to that of ZnZn-25 as can be observed in FIG. 4.

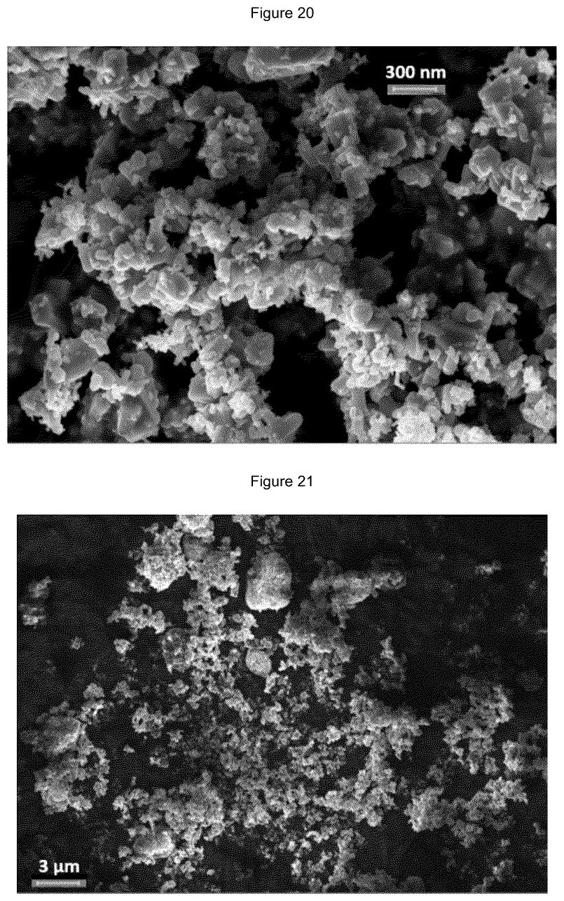

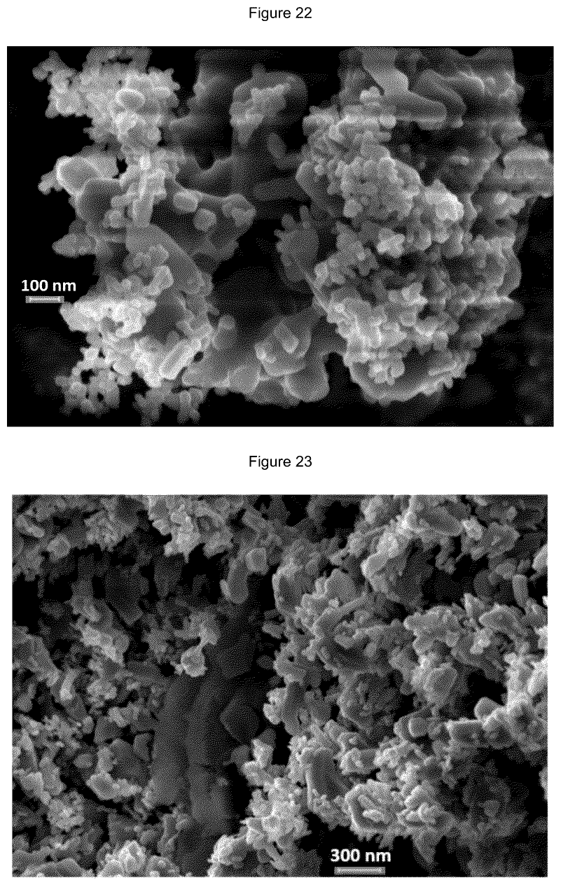

The SEM powder images were obtained as described in Examples 1-4. SEM powder images of ZnZn-argania composite as described in this example are shown in FIGS. 20-21.

SEM powder-in-ethanol image of ZnZn-argania was obtained as described in Example 1 and is shown in FIG. 22.

### Example 6: ZnZn-Caolin Composite

Another composite material was prepared by a combination of ZnZn-25 prepared as described in Example 1 (85-95% wt) and kaolin CI-77004 (8-12% wt) support particles (size lower than 53 μm) composition (SiO2 47%, Al2O3 37%, K2O 1.40%, Fe2O3 1.10% MgO 0.15%, Na2O 0.15%, TiO2 0.10%, CaO 0.05%) as starting materials. The mixture was added to the mixing chamber of a bicone mixer as described in Example 1 and stirred at a speed of 20-30 rpm for 30-120 minutes.

The results of particle size (laser diffraction), BET surface area and Z potential are shown in Table 7.

TABLE 7
Characterization of ZnZn-kaolin composite.

| | Particle size | Specific surface | Z potential ± |
|---|---|---|---|
| | D(v, 0.5) | area BET | std |
| Material | (μm) | (m²/g) | (mV) |
| ZnZn-kaolin | 6.188 | N/A | −15.20 ± 6.2 |

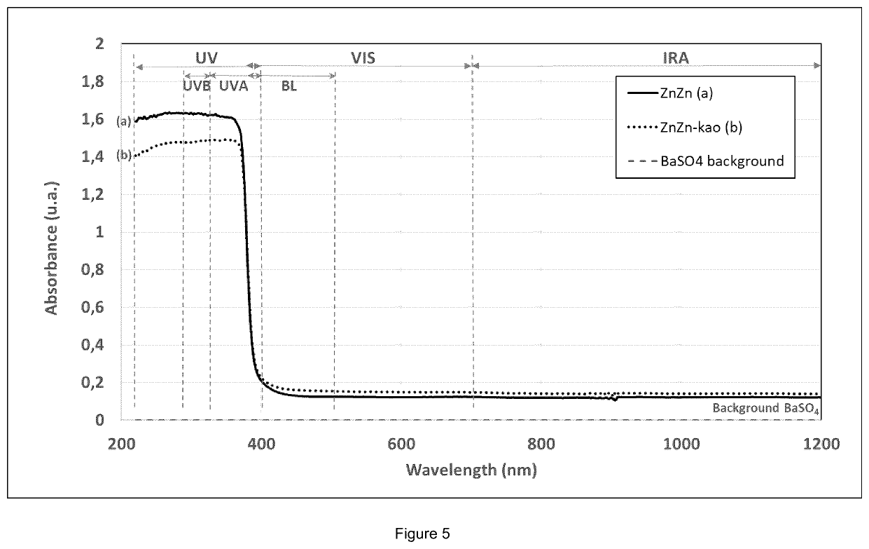

The UV-Vis-IR absorbance obtained with this material was similar to that of ZnZn-25, with lower absorbance in UV area, mainly of UVB, as can be observed in FIG. 5. Results of particle size are aligned to those of ZnZn-22.55% NP while Z potential showed the most negative values observed due to kaolin particles.

The SEM powder image was obtained as described in Examples 1-5. SEM powder image of ZnZn-caolin composite as described in this example is shown in FIG. 23.

### Example 7: SPF Data of Sunscreen Formulations

UV filters ZnZn-25 from Example 1, ZnZn-warm from Example 2, ZnZn-niacinamide, ZnZn-upcarrot, ZnZn-argania and ZnZn-caolin from Examples 3-6 were dispersed in the aqueous phase of a sunscreen cosmetic formulation with 15 wt % ZnO (INCI: Aqua, Zinc Oxide, Butylene Glycol Cocoate, Butyloctyl Salicylate, Cetearyl alcohol & Cocoglucoside, Dimethicone, Butyrospermum Parkii Butter, C14-22 Alcohol & C12-20 Alkyl Glucoside, Microcrystalline Cellulose and Cellulose Gum, Phenoxyethanol, Ethylhexylglycerin; reference named ADP17-TX). The following day (time=1 d), the SPF, UVA PF and critical wavelength (CW) values of the sunscreens were measured in vitro following the protocol as disclosed in Example 1, and are shown in Table 8.

TABLE 8
UV absorbance parameters of sunscreen formulations

| Material | SPF ± std | UVA ± std | UVA/UVB | CW |
|---|---|---|---|---|
| ZnZn-25 | 43 ± 14 | 21 ± 7 | 0.68 | 377 |
| ZnZn-color warm | 74 ± 12 | 21 ± 7 | 0.72 | 382 |
| ZnZn-niacinamide | 48 ± 1 | 23 ± 1 | 0.65 | 377 |
| ZnZn-carrot | 40 ± 1 | 20 ± 11 | 0.67 | 377 |
| ZnZn-argania | 47 ± 16 | 23 ± 7 | 0.68 | 377 |
| ZnZn-kaolin | 45 ± 9 | 22 ± 4 | 0.67 | 376 |

Overall, a synergy was observed for the ZnZn composite materials as compared with the corresponding simple mixture of ingredients ZnO micro+nanoparticles. Also when further dispersed ingredients were incorporated, as they exhibited a superior effectiveness and more prolonged stability than their corresponding simple mixture counterparts as illustrated in the Table below.

TABLE 9
UV absorbance parameters of sunscreen formulations.

| Material | SPF ± std | UVA ± std | UVA/UVB | WC | Time |
|---|---|---|---|---|---|
| ZnZn-25% | 44 ± 6 | 24 ± 3 | 0.71 | 376 | 6 m |
| ZnO micro and ZnO | 31 ± 4 | 15 ± 2 | 0.66 | 376 | 6 m |
| nano simple mixture | | | | | |
| (comparative) | | | | | |
| ZnZn-color warm | 87 ± 26 | 41 ± 10 | 0.74 | 383 | 1 m |
| ZnZn-25 + iron oxides | 45 ± 8 | 21 ± 3 | 0.73 | 382 | 1 m |
| simple mixture | | | | | |
| (comparative) | | | | | |
| ZnZn-argania | 59 ± 2 | 29 ± 1 | 0.70 | 377 | 1 m |
| ZnZn-25 + argania | 42 ± 2 | 21 ± 1 | 0.69 | 376 | 1 m |
| simple mixture | | | | | |
| (comparative) | | | | | |
| ZnZn-kaolin | 50 ± 7 | 26 ± 3 | 0.67 | 376 | 1 m |
| ZnZn-25 + kaolin | 41 ± 0 | 20 ± 5 | 0.66 | 375 | 1 m |
| simple mixture | | | | | |
| (comparative) | | | | | |

As can be observed from the table, the effectiveness of the ZnZn product remained more stable after 6 months than the simple mixture of ZnO micro+nanoparticles.

The UVA/UVB ratio of the ZnZn-warm formulation is higher than for the ZnZn material, which would indicate the iron oxides contributed to UVA protection. Besides, results were superior in protection effectiveness (SPF) to the simple mixture of ZnZn with pigments incorporated in the formulation at the same concentration (15 wt %).

### Example 8: Analysis of Stability and In Vivo Performance

Protocol when the Filter is Added to the Aqueous Phase

A formulation 1 with composition: filter+[Aqua, Butylene Glycol Cocoate, Butyloctyl Salicylate, Cetearyl alcohol & Cocoglucoside, Dimethicone, Butyrospermum Parkii Butter, C14-22 Alcohol & C12-20 Alkyl Glucoside, Microcrystalline Cellulose and Cellulose Gum, Phenoxyethanol, Ethylhexylglycerin] was selected.

The aqueous phase contains the composite: A gum (Cellulose) was previously added to the aqueous phase, containing the preservative, in a 2% wt to ensure that the UV filter remains in suspension and the SPF is homogeneous. After the rubber swells and acquires its structure which accommodates the filter, the UV filter is slowly incorporated to the aqueous phase in a 15 wt % wt under stirring and temperature (T≥60° C.). To achieve an homogeneous dispersion, the aqueous phase is stirred for 20-30 min.

The ingredients of the oil phase are melted under stirring up to 75° C. When both phases are at 75° C., oil phase is added to aqueous phase to emulsify, keeping medium stirring for 10 min. Then the emulsion is homogenized (ultra-turrax, 5 min, up to 10000-15000 rpm). After, the emulsion temperature is progressively lowered to RT, under low stirring.

A formulation 2 with composition: filter+[Aqua, Propanediol, Glycerin, Butyloctyl Salicylate, Cetearyl Alcohol, Cetearyl Glucoside, Phenoxyethanol, C14-22 Alcohols, C12-20 Alkyl Glucoside, Microcrystalline Cellulose, Cellulose Gum, Ethylhexylglycerin, Xanthan Gum] was selected.

The aqueous phase contains the composite: A gum (mixture of cellulose and xanthan) was previously added to the aqueous phase, containing the preservative and propanediol, in a 2% wt to ensure that the UV filter remains in suspension and the SPF is homogeneous. After the rubber swells and acquires its structure which accommodates the filter, the UV filter is slowly incorporated to the aqueous phase in a 17 wt % wt under stirring and temperature (T≥60° C.). To achieve a homogeneous dispersion, the aqueous phase is stirred for 20-30 min. The oil phase and emulsion preparation is performed as in the formulation 1.

The performance and stability were analyzed by measuring the transmittance expressed as SPF, UVA PF and critical wavelength (CW) values with the SPF-290AS analyzer from Solar Light. By following an internal protocol, adapted from ISO 24443 for in vitro testing, 1.3 mg/cm2 of sunscreen were spread over 50×50 mm2 PMMA plate of 5 microns-controlled roughness (Schönberg Gmbh. & Co, Germany (3 plates, 9 measurements on each plate). Analyses were performed one day after the formulation 1 preparation and after 1 month to evaluate the stability.

In vitro analyses were performed one day after the formulation 2 preparation and after 1 month to evaluate the stability of the formula. Analyses included the determination of the in vivo SPF protection factor according to ISO 24444:2019/Amd.1:2022. The results made it possible to verify that the analyses using the in vitro method adapted in the laboratory correspond to the result on human skin. In vivo determination is mandatory in order to market a cosmetic product claiming a specific SPF.

| | In vitro SPF | In vivo SPF |
|---|---|---|
| Filter | ( 1 month) | (No. volunteers) |
| ZnZn, 17% | 43 ± 0 | 39.7 ± 3.3 (3 volunteers) |
| ZnZn-nia, 17% | 35 ± 3 | 33.2 ± 5.6 (3 volunteers) |
| ZnZn-arg, 17% | 51 ± 1 | 50.3 ± 6.6 (6 volunteers) |

Protocol when the Filter is Added to the Oil Phase

A formulation 3 with composition: filter+[Aqua, Caprylic/Capric Triglyceride, Coco-Caprylate/Caprate, VP/Hexadecene Copolymer, Glycerin, Sorbitan Olivate, Magnesium sulphate, Polyglyceryl-6 Polyhydroxystearate, Polyglyceryl-6 Polyricinoleate, Polyhydroxystearic Acid, Phenoxyethanol, Stearalkonium Hectorite, Polyglyceryl-3 Diisostearate, Polyglyceryl-3 Polyricinoleate, Ethylhexylglycerin] was selected

The aqueous phase contains: Aqua, Glycerin, Magnesium sulphate, Phenoxyethanol (and) Ethylhexylglycerin. The oil phase contains the rest of ingredients that are melted under stirring at 80-85° C. before the addition of the filter. The filter (15 wt %) is added to the oil phase under stirring and temperature (20-30 min): this phase is a mixture of oils/fats containing at least one dispersing agent to avoid agglomeration. With both phases at the same temperature, aqueous phase is added to oil phase to emulsify, keeping medium stirring for 10 min. Then the emulsion is homogenized (ultra-turrax, 5 min, up to 10000 rpm). After, the emulsion temperature is progressively lowered to RT, under low stirring.

The performance and stability were analyzed by measuring the transmittance expressed as SPF, UVA PF and critical wavelength (CW) values with the SPF-290AS analyzer from Solar Light. By following an internal protocol, adapted from ISO 24443 for in vitro testing, 11.3 mg/cm2 of sunscreen were spread over 50×50 mm2 PMMA plate of 5 microns-controlled roughness (Schönberg Gmbh. & Co, (3 plates, 9 measurements on each plate). Analyses were performed one day after the formulation 3 preparation and after 1 month to evaluate the stability. The performance and stability were analyzed in vitro as indicated for formulation 1. The in vivo SPF protection factor determination was performed as for formulation 2.

| | In vitro SPF | In vivo SPF |
|---|---|---|
| Filter | ( 1 month) | (No. volunteers) |
| ZnZn-25% NP, 15% | 65 ± 2 | 46.7 ± 3.7 (6) |

### Example 9: High Resolution Microscopy of ZnZn-25 Suspended Cryogenic Transmission Electron Microscopy Cryo-TEM

Cryo-TEM images were obtained using a JEOL JEM-2011 transmission electron microscope (Jeol Ltd, Tokyo, Japan) operating at 200 kV. A small drop of the sample was placed on a glow discharged copper grid coated with a perforated polymer film (EMR Lacey Carbon support film on copper 300 square mesh). Immediately after, the grid was plunged (Leica EM CPC) into liquid ethane held at a temperature just above its freezing point (−179.15° C.). The specimens were kept cold (−196.15° C.) during both the transfer and viewing procedures. Images were recorded on a Gatan RIO 16 camera using Digital Micrograph 3.50.3584.0

A dispersion of ZnZn-25 from Example 1 in hydroxypropylmethylcellulose was prepared and diluted to be visualized by cryo-TEM analysis as follows

### Protocol of the Dispersion Preparation

In order to prepare a dispersion at approximately equal to 1% (w/v), 2.0 g of ZnZn-25 from Example 1 are added to a solution of Hydroxypropylmethylcellulose HPMC (4.0 g) in ultrapure water (197.8 g). The mixture was stirred at 450 rpm, and at a temperature of 65° C. for 15 minutes, followed by a cool down process under stirring at 450 rpm during 2 hours.

### Protocol of the Dispersion Dilution

The ZnZn-25 dispersion at 1% (0.31 g) prepared in the previous step was diluted in 1.2 mL of ultrapure water (0.31 g in 1.2 mL), to achieve a dilution factor of 5 (1 part of ZnZn-25 dispersion at 1% dissolved in 5 parts of ultrapure water). The diluted sample was then homogenized and immediately vitrified to be studied under cryo-TEM.

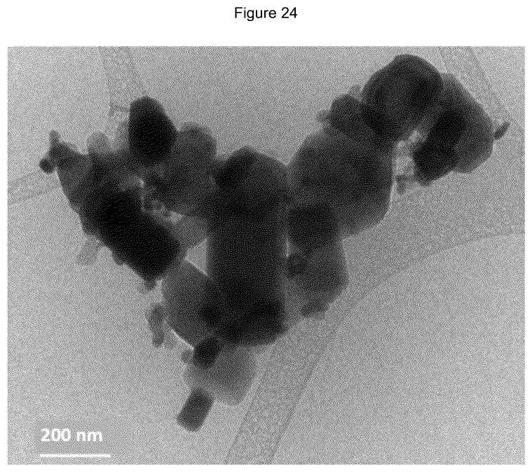

The cryo-TEM image obtained is shown in FIG. 24.

### Example 10: Anti-Inflammatory Efficacy Study of ZnZn-25, ZnZn-Niacinamide and ZnZn-Argania

Antioxidant activity was determined by anti-inflammatory efficacy study in irradiated human skin explants. Human organotypic skin explant cultures (hOSEC) were used. Distress mimicking skin inflammation was induced by daily solar-like irradiation (5 J/cm²) of skin explants for 2 days. Prior to irradiation hOSEC are topically treated with sunscreens containing 17% wt of filter concentration (2 mg/cm²). The photo-protective efficacy of the test items applied topically on hOSEC was determined by measuring pro-inflammatory cytokines (IL-6 and IL-8) by ELISA essay.

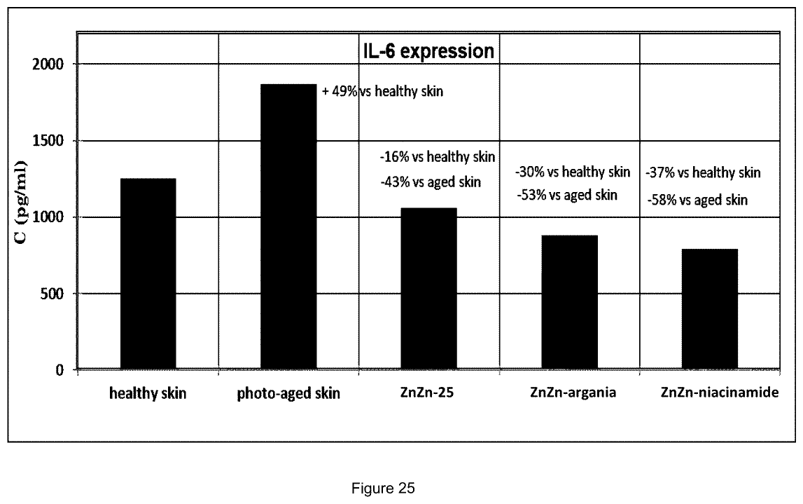

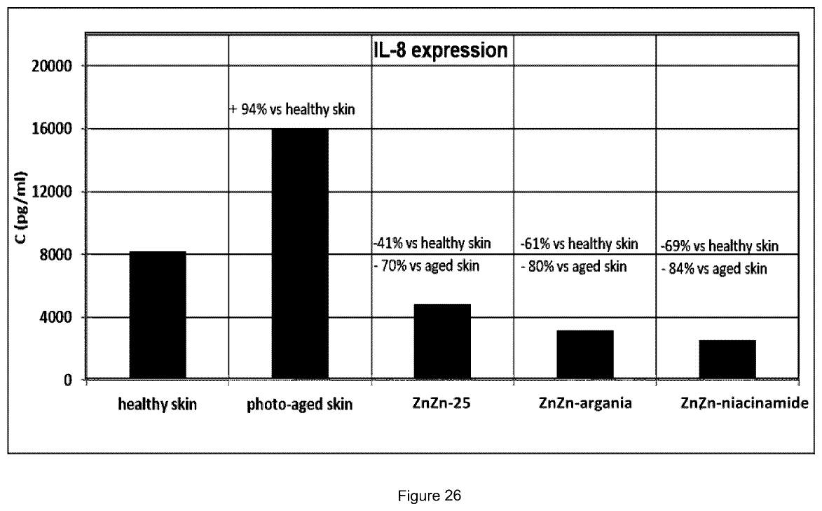

Anti-inflammatory activity was demonstrated for ZnZn-25, ZnZn-niacinamide and ZnZn-argania, by reducing IL-6 and IL-8 expression (44-58% and 69-84%, respectively, vs. photo-aged skin), as shown in FIGS. 25 and 26. Interesting results were also observed in comparison with non-irradiated (healthy) skin because all the filters showed lower secretion of pro-inflammatory cytokines.

1. A hierarchical composite material comprising:
a. ZnO nanoparticles having a mean particle size of less than 100 nm, and
b. ZnO support particles having a mean particle size between 120 and 800 nm;
wherein said nanoparticles are dispersed onto the support particles.

2. The composite material according to claim 1, wherein the weight ratio of ZnO nanoparticles:ZnO support particles in the material is from about 0.2 to 0.6, preferably from 0.25 to 0.45.

3. The composite material according to claim 1, wherein the ZnO nanoparticles have a mean particle size between 10 and 99 nm.

4. The composite material according to claim 1, wherein the ZnO support particles have a mean particle size between 150 and 600 nm.

5. The composite material according to claim 1, wherein the material further comprises iron oxide particles having a mean particle size between 10 and 2000 nm dispersed on the support particles.

6. The composite material according to claim 5, wherein the iron oxide is selected from the group consisting of FeOOH, Fe₂O₃, Fe₃O₄ and combinations thereof.

7. The composite material according to claim 5, wherein the iron oxide nanoparticles are comprised in a 0.1 to 15% by weight with respect of the total weight of the composite material.

8. The composite material according claim 1, wherein the composite material further comprises at least one organic cosmetic ingredient.

9. The composite material according to claim 1, wherein the composite material further comprises at least one organic cosmetic ingredient selected from the group consisting of niacinamide, Argania spinosa leaf extract, carrot extract and combinations thereof.

10. The composite material according to claim 1, wherein the composite material further comprises at least one inorganic cosmetic ingredient.

11. A cosmetic composition comprising the composite material according to claim 1.

12. The cosmetic composition according to claim 11, wherein the composition has a SPF of at least 25.

13. A method for the preparation of a hierarchical composite material according to claim 1, said method comprising the steps of:
(i) providing ZnO support particles with a mean size between 120 and 800 nm,
(ii) adding ZnO nanoparticles with a mean size of less than 100 nm to said support microparticles, and
(iii) stirring in a dry medium to disperse the nanoparticles onto the support particles, at a speed from 5 to 100 rpm.

14. The method according to claim 13, wherein step (ii) comprises adding at least one further cosmetic ingredient, to the support particles.

15. The method according to claim 13, wherein step (ii) comprises adding iron oxide particles to the support particles.

16. The method according to claim 13, wherein the method further comprises the steps of:
a. adding at least one further cosmetic ingredient selected from the group consisting of niacinamide, Argania spinosa extract, carrot extract, kaolin, hydroxyapatite and combinations thereof to the composite material of step (iii), and
b. stirring in a dry medium to disperse said ingredient onto the support particles of the composite material, at a speed from 5 to 100 rpm.

17. A method of filtering UVA and UVB radiation, said method comprising the topical application of the composite material as defined in claim 1 to a subject in need thereof.

18. A method of filtering UVA and UVB radiation, said method comprising the topical application of the cosmetic formulation according to claim 11 to a subject in need thereof.

19. A method of treatment or prevention of skin inflammation, said method comprising administering an effective amount of the hierarchical composite material as defined in claim 1 to a subject in need thereof.

20. The method of treatment or prevention of skin inflammation according to claim 19, wherein the hierarchical composite material further comprises at least one organic cosmetic ingredient selected from the group consisting of niacinamide, Argania spinosa leaf extract, carrot extract and combinations thereof.
